(19) United States
(12) Patent Application Publication
(10) Pub. No.: US 2018/0177713 A1
(43) Pub. Date: Jun. 28, 2018

(54) AGENT AND METHOD FOR THE TEMPORARY SHAPING OF KERATINOUS FIBERS

(71) Applicant: Henkel AG & Co. KGaA, Duesseldorf (DE)

(72) Inventors: Sandra Fuchs, Pinneberg (DE); Anna Puls, Winsen (Luhe) (DE)

(73) Assignee: Henkel AG & Co. KGaA, Duesseldorf (DE)

(21) Appl. No.: 15/847,935

(22) Filed: Dec. 20, 2017

(30) Foreign Application Priority Data
Dec. 22, 2016 (DE) 10 2016 225 960.1

(51) Int. Cl. A61K 8/92; A61K 8/81; A61K 8/34; A61Q 5/06

(52) U.S. Cl. CPC A61K 8/925; A61K 8/922; A61K 8/8141; A61K 8/342; A61K 2800/594; A61K 8/8176; A61K 8/345; A61Q 5/06; A61K 2800/48; A61K 8/8182

(57) ABSTRACT

Agents and compositions and methods are provided for the temporary shaping of keratinous fibers. In an exemplary embodiment, cosmetic composition includes from about 1.0 to about 20 wt. % wax; from about 0.1 to about 1.5 wt. % thickener; from about 1.0 to about 15 wt. % emulsifier; from about 1.0 to about 15 wt. % film-forming polymer; from about 1.0 to about 15 wt. % organic polyol; and from about 1.0 to about 90 wt. % water, all in relation to the overall weight of the cosmetic composition, wherein the weight ratio of wax to thickener is from about 40:1 to about 1:1, the utilization thereof and the method with use of this composition.

### CROSS-REFERENCE TO RELATED APPLICATION

This application claims priority to German Patent Application No. 10 2016 225 960.1, filed Dec. 22, 2016, which is incorporated herein by reference in its entirety.

### TECHNICAL FIELD

The application relates to the technical art of temporary shaping of keratinous fibers, more particularly human hair.

### BACKGROUND

Styling agents for deforming keratinous fibers have long been recognized and are used in various forms for creating, refreshing and setting hairstyles which, for many types of hair, can be achieved only using firming active ingredients. Both hair treatment agents that lend the hair a permanent shape, as well as those that lend the hair a temporary shape, play an important role here.

Sprayable products, as well as product formats that are worked into the hairstyle using a comb or the fingers, are suitable for the temporary shaping of keratinous fibers. The latter product group comprises oils, as well as waxes, gels, creams and powders.

A current trend in temporary hair shaping is the “undone look” or “out of bed look”. A hairstyle in line with this trend appears styled, without the styling agent being immediately apparent, in the form of an oily shine for example. This customer requirement can be realized first and foremost by means of sprays and powders, since such product formats usually contain only small proportions of waxes and oils. However, powders and sprays in particular cannot be applied directly to the hair by means of a comb or the fingers.

A direct application with the fingers, on the other hand, is possible in the case of wax-like hair shaping agents. Although such application offers advantages for individual hair styling, this usually requires the addition of high melting point oils and waxes, which leave behind an oily shine on the hair after use.

### BRIEF SUMMARY

Agents and compositions and methods are provided for the temporary shaping of keratinous fibers. In an exemplary embodiment, a cosmetic composition includes from about 1.0 to about 20 wt. % wax; from about 0.1 to about 1.5 wt. % thickener; from about 1.0 to about 15 wt. % emulsifier; from about 1.0 to about 15 wt. % film-forming polymer; from about 1.0 to about 15 wt. % organic polyol; and from about 1.0 to about 90 wt. % water, all in relation to the overall weight of the cosmetic composition, wherein the weight ratio of the wax to the thickener is from about 40:1 to about 1:1.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure therefore addressed the problem of providing hair treatment agents for temporary shaping, which achieve a long-lasting style and good remodeling characteristics, but which give the hairstyle a dry and oil-free appearance. In addition, it should be possible to apply the hair treatment agents to the hair using the hands.

It has been established that these tasks can be solved by means of a thickened combination of active ingredients.

The present application provides:
1. Cosmetic composition comprising, in relation to its overall weight,
a) from about 1.0 to about 20 wt. % wax
b) from about 0.1 to about 1.5 wt. % thickener
c) from about 1.0 to about 15 wt. % emulsifier
d) from about 1.0 to about 15 wt. % film-forming polymer
e) from about 1.0 to about 15 wt. % organic polyol
f) from about 1.0 to about 90 wt. % water wherein the weight ratio of wax to thickener is from about 40:1 to about 1:1.
2. Cosmetic composition according to Point 1, wherein the weight ratio of wax to thickener is from about 30:1 to about 2:1, such as from about 20:1 to about 4:1.
3. Cosmetic composition according to one of the points above, wherein the percentage by weight of wax of the total weight of the composition is from about 2.0 to about 15 wt. %, such as from about 3.0 to about 8.0 wt. %.
4. Cosmetic composition according to one of the points above, wherein the wax is selected from the group of beeswax, carnauba wax, candelilla wax, and paraffin wax, such as from the group of beeswax, carnauba wax, and mixtures thereof.
5. Cosmetic composition according to one of the points above, wherein the percentage by weight of thickener of the total weight of the composition is from about 0.1 to about 1.2 wt. %, such as from about 0.1 to about 0.9 wt. %.
6. Cosmetic composition according to one of the points above, wherein the thickener is selected from the group of polymeric organic thickeners, such as from the group of acrylic acid homo- or copolymers, for example from the group of acrylic acid homopolymers.
7. Cosmetic composition according to one of the points above, wherein the percentage by weight of emulsifier of the total weight of the composition is from about 1.5 to about 12 wt. %, for example from about 2.0 to about 10 wt. %.
8. Cosmetic composition according to one of the points above, wherein the emulsifier is selected from the group of polyalkoxylated fatty alcohols.
9. Cosmetic composition according to one of the points above, wherein the emulsifier is selected from the group of
i) Ethylene oxide adducts of saturated or unsaturated linear fatty alcohols each having from about 2 to about 50 mol of ethylene oxide per mol of fatty alcohol, for example from about 2 to about 30 mol of ethylene oxide per mol of fatty alcohol
ii) Ethylene oxide propylene oxide adducts of linear fatty alcohols with from about 8 to about 30 carbon atoms, for example the adducts of from about 2 to about 50 mol of ethylene oxide and from about 1 to about 5 mol of propylene oxide of linear fatty alcohols having from about 12 to about 18 carbon atoms
10. Cosmetic composition according to one of the points above, wherein the percentage by weight of film-forming polymer of the total weight of the composition is from about 1.0 to about 12 wt. %, such as from about 2.0 to about 10 wt. %.
11. Cosmetic composition according to one of the points above, wherein the film-forming polymer is selected from the group of non-ionic film-forming polymers.
12. Cosmetic composition according to one of the points above, wherein the film-forming polymer is selected from the group of polyvinylpyrrolidones (INCI designation: PVP) and vinylpyrrollidone/vinylacetate copolymers (INCI designation VP/VA copolymer).
13. Cosmetic composition according to one of the points above, wherein the percentage by weight of polyol of the total weight of the composition is from about 1.0 to about 10 wt. %, such as from about 1.0 to about 7.0 wt. %.
14. Cosmetic composition according to one of the points above, wherein the polyol is selected from the group of propylene glycol, glycerin and panthenol.
15. Cosmetic composition according to one of the points above, wherein the percentage by weight of water of the total weight of the composition is from about 15 to about 90 wt. %, such as from about 30 to about 90 wt. %.
16. Cosmetic composition according to one of the points above, wherein the composition additionally contains from about 1.0 to about 10 wt. %, such as from about 2.0 to about 10 wt. %, such as from about 4.0 to about 10 wt. % of one of the lipids differing from the wax.
17. Cosmetic composition according to Point 16, wherein the composition contains a lipid from the group of mineral oils, natural oils, synthetic oils or mixtures thereof.
18. Cosmetic composition according to one of the aforementioned items, wherein the composition exists in the form of paste.
19. Use of a cosmetic composition according to one of Points 1 to 14 for the temporary deformation of keratinous fibers.
20. Method for the temporary deformation of keratinous fibers, particularly human hair, wherein the cosmetic composition according to one of Points 1 to 14 is applied to keratinous fibers.

The cosmetic composition as contemplated herein preferably exists in the form of a paste.

A first essential component of the compositions as contemplated herein is the wax. Exemplary cosmetic compositions contain from about 2.0 to about 15 wt. %, such as from about 3.0 to about 8.0 wt. % wax, relative to their total weight.

The exemplary wax has a melting point in the range of from about 40° C. to about 90° C., such as from about 50° C. to about 85° C., for example from about 50° C. to about 75° C.

In principle, all waxes which melt within the stated temperature range and which are physiologically tolerable can be used. Exemplary waxes can be allocated to the groups of natural waxes, mineral waxes and synthetic waxes. Exemplary embodiments use natural waxes. Individual exemplary waxes as contemplated herein are beeswax, carnauba wax, candelilla wax and paraffin wax, such as beeswax and carnauba wax as well as mixtures thereof.

A second essential component of the cosmetic compositions as contemplated herein is the thickener. In certain embodiments, it may be beneficial with regard to the producibility, applicability and cosmetic effect of cosmetic compositions as contemplated herein if the percentage by weight of the thickener of the total weight of the cosmetic preparation is from about 0.1 to about 1.2 wt. %, such as from about 0.1 to about 0.9 wt. %.

Exemplary thickeners are selected from the group of polymeric organic thickeners. The polymeric organic thickeners can be crosslinked or non-crosslinked.

A first group of exemplary thickeners contains at least one structural unit selected from at least one structural unit having the formula (I) or salt forms thereof or at least one of a structural unit-II) or salt forms thereof,

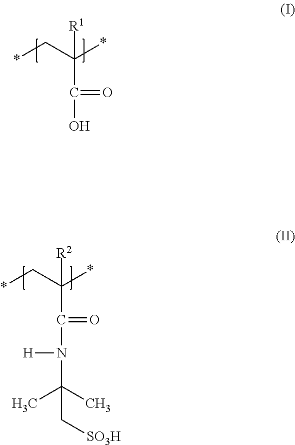

wherein R¹ and R² denote a water atom or a methyl group independent of each other,

According to the formulae above and all formulae below, a chemical compound identified with the symbol * denotes a free valence of the corresponding structural fragment.

Exemplary anionic thickening polymers contain at least one structural unit of formula (I). Exemplary thickeners are selected from the group of acrylic acid homo- or copolymers. Particular preference is given to acrylic acid homopolymers.

Exemplary thickeners are
polyacrylic acids having the INCI designation carbomer, which are sold, for example, from the company 3V Sigma under the trade name Synthalen® K or from the company Lubrizol under the trade name Carbopol.

A second exemplary group of thickeners comprises the polymeric, anionic, amphiphilic thickeners. Such thickeners may consist of at least one structural unit of the formula (III) and at least one structural unit of the formula (IV),

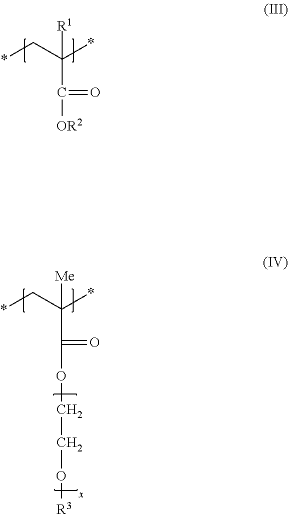

wherein
R¹ denotes a hydrogen atom or a methyl group,
R² denotes a hydrogen atom or a C₁ to C₆-alkyl group,
R³ denotes a (C₈ to C₃₀)-alkyl group,
M⁺ denotes a physiologically compatible cation and
x denotes an integer from about 0 to about 35.

Particular preference is given to the thickeners with the INCI designations Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer and Acrylates/Steareth-50 Acrylate Copolymer.

Exemplary thickeners are
thickeners having the INCI designation Acrylates/Steareth-20 Methacrylate Copolymer, such as those sold by the company Rohm&Haas under the trade name Aculyn® 22.
thickeners having the INCI designation Acrylates/Steareth-20 Methacrylate Crosspolymer, such as those sold by the company Rohm&Haas under the trade name Aculyn® 88.
thickeners having the INCI designation Acrylates/Steareth-20 Itaconate Copolymer, such as those sold by the company National Starch under the trade name Structure 2001.
   additional polymers, anionic, amphiphilic thickeners exemplified by long-chain alkyl substituents. This group includes, for example, the compounds with the INCI designations Acrylates/Stearyl Methacrylate Copolymer, Acrylates/Vinyl Isodecanoate Crosspolymer.

Exemplary thickeners are
thickeners with the INCI designation Acrylates/C₁₀₋₃₀ Alkyl Acrylate Crosspolymer, such as those sold under the trade name Carbopol Ultrez 21 by the company Lubrizol.

For the dispensing of the inventive compositions during production, their applicability and cosmetic effect, narrowly defined limits of the weight ratio of wax and thickener been found to be advantage. Weight ratios of wax to thickener of from about 30:1 to about 2:1, such as from about 20:1 to about 4:1 have been found to be particularly advantageous.

The cosmetic compositions contain an emulsifier as a third essential component. Exemplary cosmetic compositions have a percentage by weight of non-ionic emulsifier of from about 1.5 to about 12 wt. %, particularly from about 2.0 to about 10 wt. %.

With regard to the cosmetic properties and dosing properties of the compositions, non-ionic emulsifiers from the group of polyalkoxylated fatty alcohols have proved to be particularly advantageous. Non-ionic emulsifiers that are solid at room temperature (20° C.) may be used.

An exemplary group of emulsifiers is the polyalkoxylated fatty alcohols, wherein ethylene oxide adduct of saturated or unsaturated linear fatty alcohols, each with from about 2 to about 50 mol of ethylene oxide per mol of fatty alcohol, such as from about 2 to about 30 mol of ethylene oxide per mol of fatty alcohol.

Examples of emulsifiers are linear or branched, saturated or unsaturated C₁₂-C₃₀ alkanols, each of which are etherified with 2-4 ethylene oxide units per molecule. Exemplary compounds have the INCI designations Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, as well as the mixtures thereof.

A second group of exemplary emulsifiers are linear or branched, saturated or unsaturated C₁₂-C₃₀ alkanols, each of which are etherified with from about 20 to about 50 ethylene oxide units per molecule. Exemplary emulsifiers are compounds with the INCI designations Laureth-20, Laureth-25, Laureth-30, Laureth-40, Laureth-50, Myreth-20, Myreth-25, Myreth-30, Myreth-40, Myreth-50, Ceteth-20, Ceteth-25, Ceteth-30, Ceteth-40, Ceteth-50, Steareth-20, Steareth-21, Steareth-25, Steareth-30, Steareth-40, Steareth-50, Ceteareth-20, Ceteareth-25, Ceteareth-30, Ceteareth-40, Ceteareth-50, Oleth-20, Oleth-25, Oleth-30, Oleth-40 and Oleth-50.

A second group of exemplary polyalkoxylated fatty alcohols is the ethylene oxide-propylene oxide adducts to linear fatty alcohols with from about 8 to about 30 carbon atoms, in particular the adducts of from about 2 to about 50 mol of ethylene oxide and from about 1 to about 5 mol of propylene oxide to linear fatty alcohols with from about 12 to about 18 carbon atoms. One example of this type of non-ionic emulsifier is the compound with the INCI designation PPG-5-Ceteth-20.

Examples of non-ionic emulsifier systems are compounds with the INCI designations Steareth-2, Steareth-21 and PPG-5-Ceteth-20 as well as mixtures of these emulsifiers.

To further enhance their cosmetic effect, the cosmetic compositions contain a film-forming polymer. This film-forming polymer is different from the thickener described above. Preference is given to the groups of anionic or nonionic polymers. Particular preference is given to the use of film-forming, nonionic polymers. The percentage by weight of these polymers of the total weight of the cosmetic preparation may be limited to amounts between about 1.0 and about 12 wt. % and particularly from about 2.0 to about 10 wt. %.

Examples of nonionic film-forming polymers are:
vinylpyrrolidone/vinyl ester copolymers, which are sold, for example, under the trade name Luviskol (BASF). Luviskol VA 64 and Luviskol VA 73, in each case vinylpyrrolidone/vinylacetate-copolymers, may be nonionic polymers.
cellulose ethers, such as hydroxypropyl cellulose, hydroxyethyl cellulose and methylhydroxypropyl cellulose, are sold, for example, under the trade name Culminaland Benecel (AQUALON).
vinylpyrrolidone, which are sold, for example, under the name Luviskol (BASF).

On the basis of their cosmetic effect, exemplary film-forming polymers are the polyvinyl pyrrolidones (INCI designation: PVP) and the vinyl-pyrrolidone/vinyl-acetate copolymers (INCI designation VP/VA copolymer).

The inventive compositions contain an organic polyol as a fifth essential component. Exemplary compositions contain the organic polyol in amounts of from about 1.0 to about 10, such as from about 1.0 to about 7.0 wt. %, relative to the total weight.

The polyol can be used as an individual substance or in the form of polyol mixtures. Exemplary agents as contemplated herein are exemplified in that they contain less than four, such as one to three, for example only one polyol. Polyols from the group glycerin, 1,2-ethanediol, polyethylene glycols with MW>400, propanediol, butanediol, particularly 1,3 butanediol, hexanediol, particularly 1,6 hexanediol, sorbitol, threitol, erythritol, arabitol, altritol, ribitol, xylitol, galactitol, mannitol, iditol and panthenol are particularly well-suited to produce cosmetics compositions in this context. Glycerin, sorbitol and panthenol are particularly suitable organic polyols. Exemplary compositions as contemplated herein are exemplified in that they contain at least one polyol from the group propylene glycol, glycerin and panthenol, such as propylene glycol.

The addition of the polyol improved the properties compositions as contemplated herein during storage and application and provides beneficial cosmetic effects.

The cosmetic agents as contemplated herein are based on an aqueous carrier system. The percentage by weight of the water of the total weight of the exemplary cosmetic composition is from about 15 to about 90 wt. %, such as from about 30 to about 90 wt. %.

In summary, the following exemplary cosmetic compounds are contained, relative to their total weight:
a) from about 2.0 to about 15 wt. % wax from the group of beeswax, carnauba wax, and mixtures thereof;
b) from about 0.1 to about 1.2 wt. % thickener from the group of acrylic acid homopolymers;
c) from about 1.5 to about 12 wt. % emulsifier from the group of:
   i) Ethylene oxide adducts of saturated or unsaturated linear fatty alcohols each having from about 2 to about 50 mol of ethylene oxide per mol of fatty alcohol, particularly from about 2 to about 30 mol of ethylene oxide per mol of fatty alcohol;
   ii) Ethylene oxide-propylene oxide adducts to linear fatty alcohols with 8 to 30 carbon atoms, in particular the adducts of from about 2 to about 50 mol of ethylene oxide and from about 1 to about 5 mol of propylene oxide to linear fatty alcohols with from about 12 to about 18 carbon atoms;
d) from about 1.0 to about 25 wt. % film-forming polymer from the group of nonionic film-forming polymers;
e) from about 1.0 to about 7.0 wt. % organic polyol from the group propylene glycol, glycerin and panthenol; and
f) from about 30 to about 90 wt. % water,
   wherein the weight ratio of wax to thickener is from about 40:1 to about 1:1.

In addition to the essential components described above, the cosmetic compositions can contain further optional active substances or excipients.

A first group of optional active ingredients and excipients are lipids. These lipids differ from the mandatory waxes described above. The percentage by weight of lipids of the total weight of the preparation may be from about 1.0 to about 10 wt. %, such as from about 2.0 to about 10 wt. %, for example from about 4.0 to about 10 wt. %.

A group of exemplary lipids includes the oils from the group of mineral oils, natural oils, synthetic oils and mixtures thereof.

Suitable oils as contemplated herein may be selected from the esters of linear or branched saturated or unsaturated fatty alcohols with from about 2 to about 30 carbon atoms having linear or branched saturated or unsaturated fatty alcohols having from about 2 to about 30 carbon atoms which can be hydroxylated. This includes cetyl-2-ethylhexanoate, 2-hexyldecylstearate (e.g. Eutanol® G 16 S), 2-hexyldecyllaurate, isodecylneopentanoate, isononylisononanoate, 2-ethylhexylpalmitate (e.g. Cegesoft® C 24) and 2-ethylhexylstearate (e.g. Cetiol® 868). Preference may be given to isopropylmyristate, isopropylpalmitate, isopropylstearate, isopropylisostearate, isopropyloleate, isooctylstearate, isononylstearate, isocetylstearate, isononylisononanoate, isotridecylisononanoate, cetearylisononanoate, 2-ethylhexyllaurate, 2-ethylhexylisostearate, 2-ethylhexylcocoate, 2-octyldodecylpalmitate, butyloctanoic acid-2-butyloctanoate, diisotridecylacetate, n-butylstearate, n-hexyllaurate, n-decyloleate, oleyloleate, oleylerucate, erucyloleate, erucylerucate, ethylene glycoldioleate and ethylene glycoldipalmitate. In certain embodiments, the cetyl-2-ethylhexanoate is used.

Additional exemplary oils as contemplated herein are selected from the benzoic acid esters of linear or branched C8-22-alkanols. Particular preference may be given to benzoic acid-C12-C15-alkyl esters, e.g., available as the commercial product Finsolv® TN, benzoic acid isostearyl esters, e.g., available as the commercial product Finsolv® SB, ethylhexylbenzoate, e.g., available as the commercial product Finsolv® EB, and benzoic acid octyldocecyl esters, e.g., available as the commercial product Finsolv® BOD.

Further optional active substances or excipients may include additional nourishing agents.

As the nourishing agent, the agent can contain at least one protein hydrolysate and/or a derivate thereof, for example. Protein hydrolysates are product mixtures obtained through the acidically, basically or enzymatically catalyzed decomposition of proteins. As contemplated herein, the expression protein hydrolysates also includes total hydrolysates, as well as individual amino acids and the derivatives thereof, as well as mixtures of various amino acids. The molecular weight of the protein hydrolysates usable as contemplated herein is from about 75, the molecular weight for glycine, to about 200,000, such as from about 75 to about 50,000, for example from about 75 to about 20,000 Dalton.

The agent as contemplated herein can also contain, as a nourishing agent a vitamin, a provitamin, a vitamin precursor and/or a derivative thereof. As contemplated herein, such vitamins, provitamins and vitamin precursors are usually assigned to the groups A, B, C, E, F and H.

Other nourishing agents are caffeine and nicotinamide.

The agent as contemplated herein can also contain, as a nourishing agent, a plant extract, as well as mono- and/or oligosaccharides and/or lipids.

The composition of some additional exemplary cosmetic compositions with which the weight ratio of wax to thickener is from about 40:1 to about 1:1, such as from about 30:1 to about 2:1, for example from about 20:1 to about 4:1, can be found in the following table (specified in wt. %, relative to the total weight of the cosmetic agent, unless otherwise indicated).

| | Formula 1 | Formula 2 | Formula 3 | Formula 4 | Formula 5 |
|---|---|---|---|---|---|
| Wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 6 | Formula 7 | Formula 8 | Formula 9 | Formula 10 |
| Beeswax, carnauba wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 11 | Formula 12 | Formula 13 | Formula 14 | Formula 15 |
| Wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 16 | Formula 17 | Formula 18 | Formula 19 | Formula 20 |
| Wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Polyalkoxylated fatty | 1.0 to 15 | 1.5 to 12 | 1.5 to 12 | 2.0 to 10 | 2.0 to 10 |
| alcohol | | | | | |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 21 | Formula 22 | Formula 23 | Formula 24 | Formula 25 |
| Wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Vinylpyrrolidone homo- | 1.0 to 15 | 1.0 to 12 | 1.0 to 12 | 1.0 to 12 | 2.0 to 10 |
| or copolymer | | | | | |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 26 | Formula 27 | Formula 28 | Formula 29 | Formula 30 |
| Wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 31 | Formula 32 | Formula 33 | Formula 34 | Formula 35 |
| Beeswax, carnauba wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 36 | Formula 37 | Formula 38 | Formula 39 | Formula 40 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 41 | Formula 42 | Formula 43 | Formula 44 | Formula 45 |
| Beeswax, carnauba wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Polyalkoxylated fatty | 1.0 to 15 | 1.5 to 12 | 1.5 to 12 | 2.0 to 10 | 2.0 to 10 |
| alcohol | | | | | |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 46 | Formula 47 | Formula 48 | Formula 49 | Formula 50 |
| Beeswax, carnauba wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Vinylpyrrolidone homo- | 1.0 to 15 | 1.0 to 12 | 1.0 to 12 | 1.0 to 12 | 2.0 to 10 |
| or copolymer | | | | | |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 51 | Formula 52 | Formula 53 | Formula 54 | Formula 55 |
| Beeswax, carnauba wax | 1.0 to 20 | 2.0 to 15 | 2.0 to 15 | 3.0 to 8.0 | 3.0 to 8.0 |
| Thickener | 0.1 to 1.5 | 0.1 to 1.2 | 0.1 to 1.2 | 0.1 to 1.2 | 0.1 to 0.9 |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 56 | Formula 57 | Formula 58 | Formula 59 | Formula 60 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Polyalkoxylated fatty | 1.0 to 15 | 1.5 to 12 | 1.5 to 12 | 2.0 to 10 | 2.0 to 10 |
| alcohol | | | | | |
| Film-forming polymer | 1.0 to 15 | 1.0 to 12 | 1.0 to 12 | 1.0 to 12 | 2.0 to 10 |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 61 | Formula 62 | Formula 63 | Formula 64 | Formula 65 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Emulsifier | 1.0 to 15 | 1.5 to 12 | 1.5 to 12 | 2.0 to 10 | 2.0 to 10 |
| Vinylpyrrolidone homo- | 1.0 to 15 | 1.0 to 12 | 1.0 to 12 | 1.0 to 12 | 2.0 to 10 |
| or copolymer | | | | | |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 66 | Formula 67 | Formula 68 | Formula 69 | Formula 70 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Emulsifier | 1.0 to 15 | 1.5 to 12 | 1.5 to 12 | 2.0 to 10 | 2.0 to 10 |
| Film-forming polymer | 1.0 to 15 | 1.0 to 12 | 1.0 to 12 | 1.0 to 12 | 2.0 to 10 |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 71 | Formula 72 | Formula 73 | Formula 74 | Formula 75 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Polyalkoxylated fatty | 1.0 to 15 | 1.5 to 12 | 1.5 to 12 | 2.0 to 10 | 2.0 to 10 |
| alcohol | | | | | |
| Vinylpyrrolidone homo- | 1.0 to 15 | 1.0 to 12 | 1.0 to 12 | 1.0 to 12 | 2.0 to 10 |
| or copolymer | | | | | |
| Polyol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 76 | Formula 77 | Formula 78 | Formula 79 | Formula 80 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Polyalkoxylated fatty | 1.0 to 15 | 1.5 to 12 | 1.5 to 12 | 2.0 to 10 | 2.0 to 10 |
| alcohol | | | | | |
| Film-forming polymer | 1.0 to 15 | 1.0 to 12 | 1.0 to 12 | 1.0 to 12 | 2.0 to 10 |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 81 | Formula 82 | Formula 83 | Formula 84 | Formula 85 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Polyalkoxylated fatty | 1.0 to 15 | 1.5 to 12 | 1.5 to 12 | 2.0 to 10 | 2.0 to 10 |
| alcohol | | | | | |
| Vinylpyrrolidone homo- | 1.0 to 15 | 1.0 to 12 | 1.0 to 12 | 1.0 to 12 | 2.0 to 10 |
| or copolymer | | | | | |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |
| | Formula 81 | Formula 82 | Formula 83 | Formula 84 | Formula 85 |
| Beeswax, carnauba wax | 1.0 to 20 | 2.0 to 15 | 2.0 to 15 | 3.0 to 8.0 | 3.0 to 8.0 |
| Acrylic acid | 0.1 to 1.5 | 0.1 to 1.2 | 0.1 to 1.2 | 0.1 to 1.2 | 0.1 to 0.9 |
| homopolymer | | | | | |
| Polyalkoxylated fatty | 1.0 to 15 | 1.5 to 12 | 1.5 to 12 | 2.0 to 10 | 2.0 to 10 |
| alcohol * | | | | | |
| Vinylpyrrolidone/ | 1.0 to 15 | 1.0 to 12 | 1.0 to 12 | 1.0 to 12 | 2.0 to 10 |
| Vinylacetate-Copolymer | | | | | |
| Propylene glycol | 1.0 to 15 | 1.0 to 10 | 1.0 to 10 | 1.0 to 7.0 | 1.0 to 7.0 |
| Water | 1.0 to 90 | 15 to 90 | 30 to 90 | 30 to 90 | 30 to 90 |
| Optional additives | ad 100 | ad 100 | ad 100 | ad 100 | ad 100 |

* From the group Steareth-2, Steareth-21 and PPG-5-Ceteth-20

As stated at the outset, the cosmetic compositions as contemplated herein are exemplified by particular advantages in terms of the temporary reshaping of keratinous fibers. A corresponding use of this cosmetic composition for temporary reshaping of keratinous fibers, especially human hair, is therefore a further object of this application.

A final object of this application is a method for the temporary reshaping of keratinous fibers, especially human hair, in which the cosmetic composition is applied to keratinous fibers.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition comprising:
a) from about 1.0 to about 20 wt. % wax;
b) from about 0.1 to about 1.5 wt. % thickener;
c) from about 1.0 to about 15 wt. % emulsifier;
d) from about 1.0 to about 15 wt. % film-forming polymer;
e) from about 1.0 to about 15 wt. % organic polyol; and
f) from about 1.0 to about 90 wt. % water, all in relation to the overall weight of the cosmetic composition,
wherein the weight ratio of the wax to the thickener is from about 40:1 to about 1:1.

2. The cosmetic composition according to claim 1, wherein the weight ratio of wax to thickener is from about 30:1 to about 2:1.

3. The cosmetic composition according to claim 1, wherein the percentage by weight of wax of the total weight of the composition is from about 2.0 to about 15 wt. %.

4. The cosmetic composition according to claim 1, wherein the percentage by weight of thickener of the total weight of the composition is from about 0.1 to about 1.2 wt. %.

5. The cosmetic composition according to claim 1, wherein the percentage by weight of emulsifier of the total weight of the composition is from about 1.5 to about 12 wt. %.

6. The cosmetic composition according to claim 1, wherein the percentage by weight of film-forming polymer of the total weight of the composition is from about 1.0 to about 12 wt. %.

7. The cosmetic composition according to claim 1, wherein the percentage by weight of polyol of the total weight of the composition is from about 1.0 to about 10 wt. %.

8. The cosmetic composition according to claim 1, wherein the percentage by weight of water of the total weight of the composition is from about 15 to about 90 wt. %.

9. Use of a cosmetic preparation according to claim 1 for temporary deformation of keratinous fibers.

10. Method for temporary deformation of keratinous fibers where the cosmetic preparation according to claim 1 is applied on the keratinous fibers.

11. The cosmetic composition according to claim 1, wherein the weight ratio of wax to thickener is from about 20:1 to about 4:1.

12. The cosmetic composition according to claim 1, wherein the percentage by weight of wax of the total weight of the composition is from about 3.0 to about 8.0 wt. %.

13. The cosmetic composition according to claim 1, wherein the percentage by weight of thickener of the total weight of the composition is from about 0.1 to about 0.9 wt. %.

14. The cosmetic composition according to claim 1, wherein the percentage by weight of emulsifier of the total weight of the composition is from about 2.0 to about 10 wt. %.

15. The cosmetic composition according to claim 1, wherein the percentage by weight of film-forming polymer of the total weight of the composition is from about 2.0 to about 10 wt. %.

16. The cosmetic composition according to claim 1, wherein the percentage by weight of polyol of the total weight of the composition is from about 1.0 to about 7.0 wt. %.

17. The cosmetic composition according to claim 1, wherein the percentage by weight of water of the total weight of the composition is from about 30 to about 90 wt. %.
